(19) United States
(12) Patent Application Publication
(10) Pub. No.: US 2024/0038373 A1
(43) Pub. Date: Feb. 1, 2024

(54) MACHINE LEARNING CLOUD BASED CARE PAIRING TECHNOLOGY AND ASSOCIATED METHODS THEREOF

(71) Applicant: Loren Martin, GLENDORA, CA (US)

(72) Inventor: Loren Martin, GLENDORA, CA (US)

(21) Appl. No.: 18/350,663

(22) Filed: Jul. 11, 2023

Related U.S. Application Data
(60) Provisional application No. 63/368,439, filed on Jul. 14, 2022.

(51) Int. Cl. G16H 40/20

(52) U.S. Cl. CPC G16H 40/20

(57) ABSTRACT

This patent describes an innovative cloud-based system that leverages machine learning algorithms to predict and facilitate the establishment of strong therapeutic alliances between care providers and help seekers. The system processes demographic, personality trait, and characteristic variables of both care providers and help seekers. These variables are used as inputs for a machine learning model that ranks care providers based on their predicted ability to establish a positive therapeutic alliance with a specific help seeker.

The term “Therapeutic Alliance” refers to a collaborative relationship between a help seeker and a help giver, such as therapists, coaches, nurses, mentors, and others. The system is applicable to various help giver-help seeker relationships, including mental health providers and clients.

The cloud-based system provides scalability, flexibility, and cost-effectiveness by using cloud computing resources. It employs a modular micro-services architecture, making it easy to develop, update, and deploy independently.

This technology revolutionizes the process of matching care providers with help seekers, ultimately improving the quality of care and support in various domains, including mental health, coaching, and mentoring.

### TECHNICAL FIELD

The present disclosure relates to computing technology for improving mental health services, and more particularly, to providing cloud-based, machine learned models for pairing help seekers with help givers or care providers.

### BACKGROUND

Outside of a help seeker's personal characteristics, the therapeutic alliance, also referred to as a working alliance between a help seeker (also referred to as a patient or a client) and a care provider (also referred to as a help giver, mental health provider or mental health professional) is an important predictor of a positive outcome in psychotherapy (Crits-Christoph, O., Connolly Gibbons, M. B., Hamilton, J., Ring-Kurtz, S., & Gallop, R. (2011). Many help seekers quit therapy after limited sessions, often after only a single session. One reason for this is a perceived “lack of fit” with the mental health provider. Therefore, there is a need for efficient computing technology that can identify patterns to optimize pairing of a help seeker and a mental health provider based upon their collaborative relationship. Continuous efforts are being made to develop such technology.

### BRIEF DESCRIPTION OF THE DRAWINGS

The various features of the present disclosure will now be described with reference to the drawings of the various aspects disclosed herein. In the drawings, the same components may have the same reference numerals. The illustrated aspects are intended to illustrate, but not to limit the present disclosure. The drawings include the following Figures:

FIG. 1 shows an example of an operating environment for the various aspects disclosed herein;

FIG. 2 shows an example of a training process used to develop a predictive machine learned model, according to one aspect of the present disclosure;

FIG. 3 shows an example of a screening process to screen care providers, according to one aspect of the present disclosure;

FIG. 4 shows an example of a pairing process to pair help seekers with care providers, according to one aspect of the present disclosure;

FIG. 5 shows an example of a cloud-based architecture, according to one aspect of the present disclosure;

FIG. 6 shows an example of inputs used to develop the predictive model, according to one aspect of the present disclosure; and

FIG. 7 shows an example of a processing system, used according to one aspect of the present disclosure.

### DETAILED DESCRIPTION

As a preliminary note, the terms “component”, “module”, “system,” and the like as used herein are intended to refer to a computer-related entity, either software-executing general-purpose processor, hardware, firmware and a combination thereof. For example, a component may be, but is not limited to being, a process running on a hardware-based processor, a hardware processor, an object, an executable, a thread of execution, a program, and/or a computer.

By way of illustration, both an application running on a server and the server can be a component. One or more components may reside within a process and/or thread of execution, and a component may be localized on one computer and/or distributed between two or more computers. Also, these components can execute from various non-transitory, computer readable media having various data structures stored thereon. The components may communicate via local and/or remote processes such as in accordance with a signal having one or more data packets (e.g., data from one component interacting with another component in a local system, distributed system, and/or across a network such as the Internet with other systems via the signal).

Computer executable components of the innovative technology disclosed herein can be stored, for example, at a non-transitory, computer readable media including, but not limited to, an ASIC (application specific integrated circuit), CD (compact disc), DVD (digital video disk), ROM (read only memory), storage class memory, solid state drive, floppy disk, hard disk, EEPROM (electrically erasable programmable read only memory), or any other storage device, in accordance with the claimed subject matter.

In one aspect, innovative technology is disclosed herein that uses patterns from machine learning algorithms including demographic, personality trait and/or characteristic variables of a client and a care provider to predict an output of a strong therapeutic/working alliance.

The term “Therapeutic Alliance” (also referred to as a working alliance or alliance) as used herein mean a collaborative relationship, e.g., between a client and a therapist. The alliance defines a collaborative relationship between a help seeker and help giver where there is an agreement on goals, assignment of tasks, and bond development. The alliance is often described between clients and therapists, but it is applicable between other help seeker and help giver relationships including athletes and coaches, patients and care givers, mentees and mentors, students and teachers, and others. Therefore, the terms help giver, mental health provider, care provider includes teachers, coaches, nurses, doctors, mentors, psychologists, and others. The term client includes any help seeker or one who needs mental health services.

The term “Personality Trait” as used herein means a relatively stable, consistent, and enduring internal characteristic that is inferred from a pattern of behaviors, attitudes, feelings, and habits in the individual as defined by the American Psychological Association (APA).

The term “Characteristic(s)” as used herein is a particular feature or quality of a person, especially any of the enduring qualities or traits that define an individual's nature or personality in relation to others as defined by the APA.

In one aspect of the present disclosure, cloud-based computing technology runs machine learning algorithms with inputs of personal attributes of help seekers and help givers to determine a combination of attributes that predict the development of a strong working alliance. The term “helping conversation feedback” as used herein is any feedback from a help seeker that can be used as a proxy for measuring the working alliance, e.g., as defined by Bordin, E. S. (1979) in “The generalizability of the psychoanalytic concept of the working alliance” in Psychotherapy: Theory, Research Practice, 16(3), 252-260. The innovative machine learning model uses help seeker inputs including the Ten-Item-Personality-Inventory (TIPI) (Gosling, S. D., Rentfrow, P. J., & Swann, W. B., Jr. (2003). A very brief measure of the Big Five personality domains. Journal of Research in Personality, 37, 504-528.),

and demographic variables together with help giver inputs including the Analogue to Multiple Broadband Inventories (AMBI) personality inventory (Yarkoni, T. (2010). The abbreviation of personality, or how to measure 200 personality scales with 200 items. Journal of Research in Personality, 44, 180-198.), Counselor Activity Self-Efficacy Scales (CASES) (Lent, R. W., Hill, C. E., & Hoffman, M. A. (2003). Development and validation of the Counselor Activity Self-Efficacy Scales. Journal of Counseling Psychology, 50(1), 97-108.), Adult Attachment Questionnaire (AAQ) (Simpson, J. A., Rholes, W. S. & Phillips, D. (1996). Conflicts in close relationships: An attachment perspective. Journal of Personality and Social Psychology, 71(5), 899-914.), and demographic variables to rank help givers based upon predicted strength of the working alliance with a specific help seeker measured by the Session Alliance Inventory (SAI) (Falkenström, F., Hatcher, R. L., Skjulsvik, T., Holmqvist Larsson, M., Holmqvist, R. (2015). Development and validation of a 6-item working alliance questionnaire for repeated administrations during psychotherapy. Psychological Assessment, 27(1), 169-183.). Details of the innovative technology/processes are provided below.

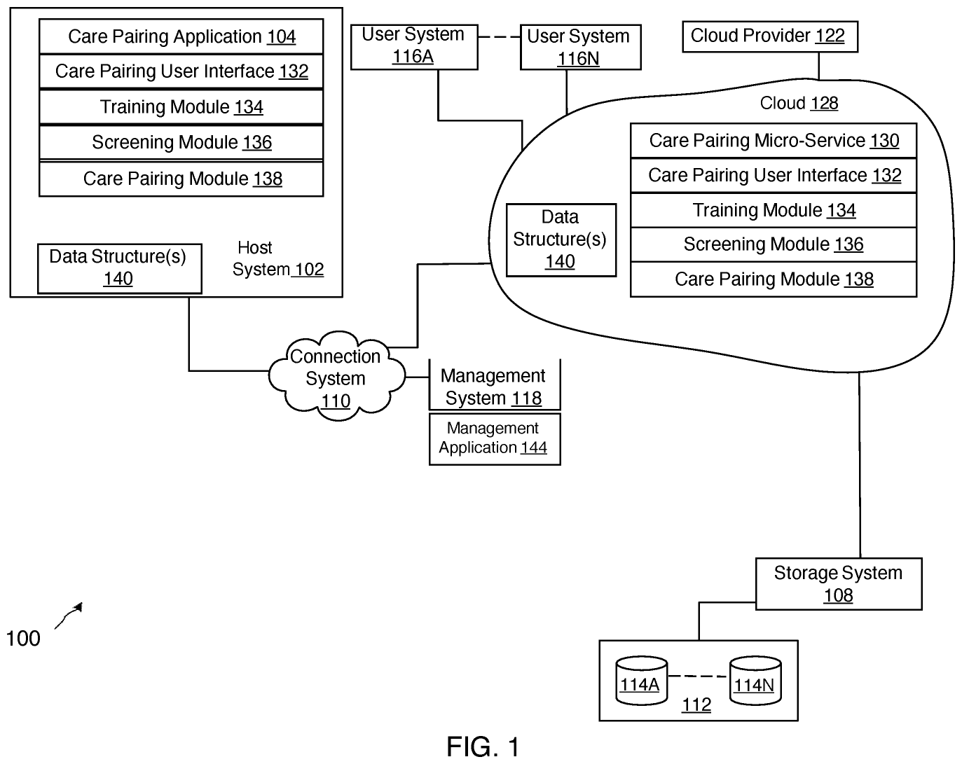

System 100: FIG. 1 shows an example of a system 100, for presenting care pairing micro-services (or microservices, used interchangeably) via a cloud-based system (or “cloud”) 128, according to one aspect of the present disclosure. Cloud 128 provides an abstraction between computing resource and its underlying technical architecture (e.g., servers, storage, networks), enabling convenient, on-demand network access to a shared pool of configurable computing resources that can be rapidly provisioned and released with minimal management effort or service provider interaction. The term “cloud” is intended to refer to a network, for example, the Internet and cloud computing allows shared resources, for example, software and information to be available, on-demand, like a public utility.

Typical cloud computing providers deliver common business applications online which are accessed from another web service or software like a web browser, while the software and data are stored remotely on servers. The cloud computing architecture uses a layered approach for providing application services. A first layer is an application layer that is executed at client computers. In this example, the application allows a client to access storage via a cloud. After the application layer, is a cloud platform and cloud infrastructure, followed by a “server” layer that includes hardware and computer software designed for cloud specific services.

In one aspect, a cloud provider 122 manages access to cloud based services for user systems/devices (or “clients”) 116A-116N that are able to access micro-services, e.g., a care pairing micro-service 130 (may also be referred to as a “micro-service 130” or micro-services 130), described below in detail. User systems 116A-116N include help giver and help seeker systems. Each micro-service 130 includes a User Interface (UI) 132, a training module 134 (also referred to as module 134), a screening module 136 (also referred to as module 136) and a care pairing module 138 (also referred to as module 138). The modules 134, 132 and 18 use one or more application programming interface (“API”) and one or more data structures 140 to execute the innovative care pairing technology of the present disclosure. The term micro-service as used herein denotes computing technology for providing a specific functionality in the networked environment.

System 100 includes a storage system 108 that may be used to store data structures 140 at one or more physical storage sites to store data. A management system 118 executing an application 144 may be used to configure the various components of system 100.

In one aspect, storage system 108 has access to a set of mass storage devices 114A-114N (may also be referred to as storage devices 114) within at least one storage subsystem 112. The mass storage devices 114 may include writable storage device media such as magnetic disks, video tape, optical, DVD, magnetic tape, non-volatile memory devices for example, solid state drives (SSDs) including self-encrypting drives, flash memory devices and any other similar media adapted to store information. The storage devices 114 may be organized as one or more groups of Redundant Array of Independent (or Inexpensive) Disks (RAID). The various aspects disclosed are not limited to any particular storage device type or storage device configuration.

The storage system 108 may be used to store and manage information (e.g. data structures 140) at storage devices 114 based on client requests. The requests may be based on file-based access protocols, for example, the Common Internet File System (CIFS) protocol or Network File System (NFS) protocol, over the Transmission Control Protocol/Internet Protocol (TCP/IP). Alternatively, the request may use block-based access protocols, for example, the Small Computer Systems Interface (SCSI) protocol encapsulated over TCP (iSCSI) and SCSI encapsulated over Fibre Channel (FCP). The adaptive aspects described herein are not limited to any specific request type or request protocol.

In one aspect, system 100 may include one or more computing systems 102 (may also be referred to as host platform(s)/system(s) 102 or simply as server(s) 102) communicably coupled to storage system 108 via a connection system 110 such as a local area network (LAN), wide area network (WAN), the Internet and others. As described herein, the term “communicably coupled” may refer to a direct connection, a network connection, or other connections to enable communication between devices. It is noteworthy that although connection system 110 and cloud 128 are shown as separate entities, functionally, the two systems may be similar in terms of providing access to host systems i.e., the host systems may access the storage systems via cloud 128.

Host system 102 stores instructions in a memory to execute a care pairing application 104 (may also be referred to as application 104) that is like the care pairing micro-service 130 with access to data structures 140. Application 104 and micro-service 130 are referred to interchangeably throughout this specification.

FIG. 1 provides an example of an architecture that enables building complex micro-services application 130 by decomposing them into a set of independent and loosely coupled services. Each service (typically running as one or more operating system processes) provides a specific capability or function and communicates with the other services over a network. Micro-service 130 of the present disclosure is modular, lightweight, and isolated. Microservice 130 can be developed independently and updated or replaced as needed. As an example, micro-service 130 may be deployed as a container (e.g., a “Docker” container described below), is stateless in nature, may be exposed as a API and is discoverable by other services.

The following provides a brief definition of various technologies used for developing and deploying micro-service 130:

“Docker”: Docker is a software framework for building and running micro-services using the Linux kernel (without derogation of any third party trademark rights). The various aspects described herein are not limited to the Linux kernel. In one aspect, Docker micro-service code for micro-service 130 is packaged as a “Docker image file”. A Docker container is then initialized using an associated image file. A Docker container is an active or running instantiation of a Docker image. Each Docker container provides isolation and resembles a lightweight virtual machine. It is noteworthy that many Docker containers can run simultaneously in a same Linux computing system.

Cloud 128 may be based on the Amazon Web Services (AWS) (without derogation of any trademark rights). However, the adaptive aspects may be implemented using other cloud platforms e.g., GOOGLE CLOUD, AZURE and others (without derogation of any third party trademark rights). AWS provides a collection of on-demand public cloud services in different categories, including compute, storage, and database services.

Cloud 128 may also leverage an “AWS Elastic Compute Cloud” that may also be referred to as “AWS EC2”, a service providing scalable compute capacity in the AWS cloud. Micro-service 130 in AWS EC2 is packaged and deployed as an Amazon Machine Image (AMI) file. In one aspect, cloud 128 may include the use of an AWS Virtual Private Cloud (VPC), a service that enables configuration of a logically isolated section of the AWS cloud to simulate a datacenter private cloud.

In one aspect, “Node.js” may be used as a “JavaScript” runtime environment for developing the micro-service 130. AngularJS may be used as a development framework for front-end web applications using Javascript. Code development for micro-service 130 may be controlled using Git repositories. It is noteworthy that these enabling technology examples are simply an illustration. The adaptive aspects of the present disclosure may be implemented using other technologies.

In one aspect, system 100 provides a platform for building and deploying care pairing technology via the cloud. The solution is flexible and extensible, lightweight, rapidly developed, low cost, and adheres to industry standards.

In one aspect, micro-service 130 may exist as an isolated repository, e.g., a local “Git” repository. Git provides technology for developing software code. Git stores data in a data structure called a repository that includes “committed” objects and references to committed objects, called “heads.” When micro-service 130 is ready to be deployed, the local Git repository is pushed to a remote repository for ongoing source code management.

In one aspect, micro-service 130 has its own unique microservices code, which is deployed as several “Docker” containers in a virtual machine. The code for each service can be derived from the samples in the repository. As an example, each service uses a dedicated AWS EC2 virtual machine.

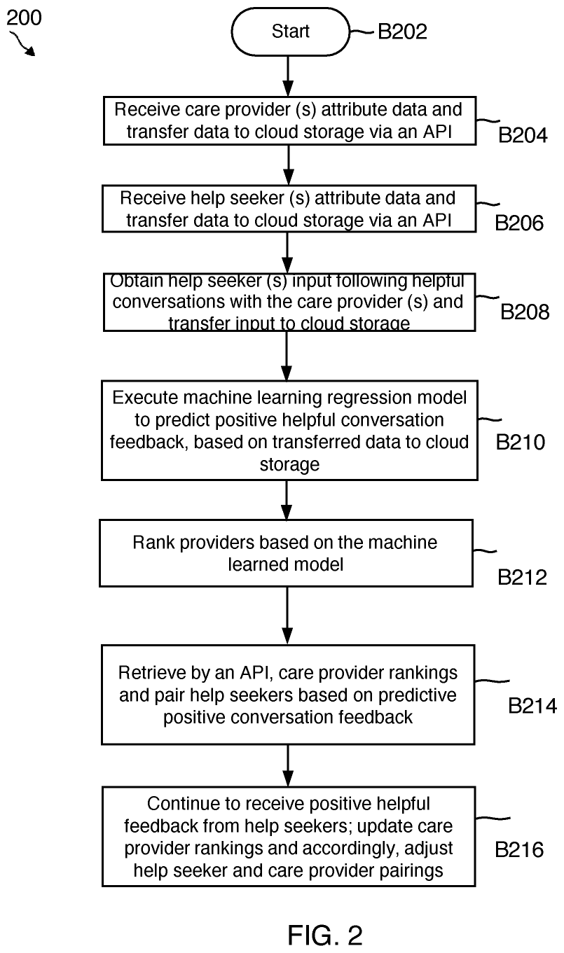

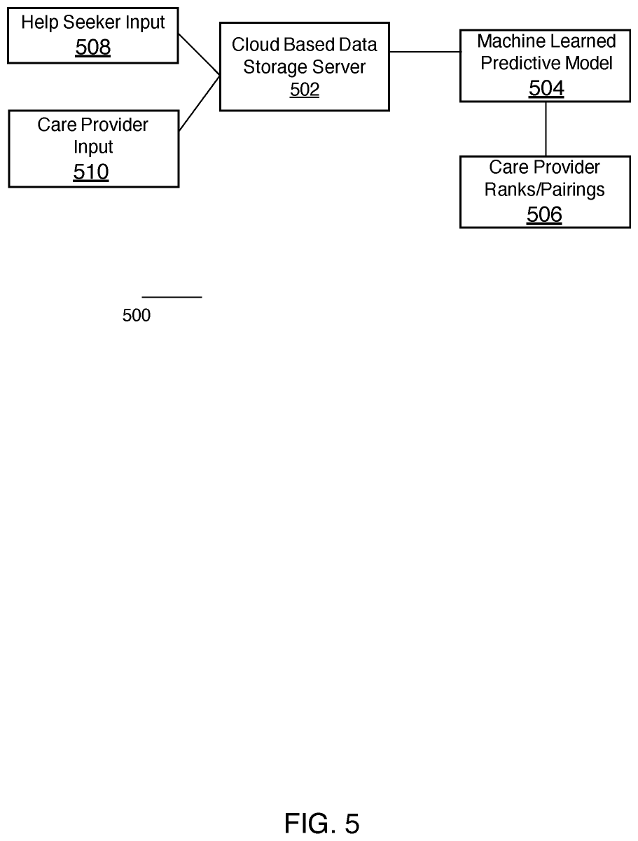

Process Flows: FIG. 2 shows a process 200 for training a machine learned model (e.g., 504, FIG. 5), according to one aspect of the present disclosure. The process begins in block B202, when a computing device (e.g., host 102)/cloud-based system has been initialized to execute the training module 134. In block B204, care provider personality attributes (e.g., 510, FIG. 5) are received and then transferred to cloud-based storage (e.g., to be stored in data structures 140) by an API. The attributes may be received by the host system 102 or directly by the micro-service 130. In block B206, help seeker attributes (e.g., 508, FIG. 5) are received and transferred to cloud-based storage via an API. The attributes may be received by the host system 102 or directly by the micro-service 130.

In block B208, help seeker input is received following helpful conversations with one or more help seekers and then transferred to cloud-based storage (e.g., to be stored in data structures 140) by an API.

In block B210, a machine learning regression model is executed to predict positive helping conversation feedback. As an example, the model is executed by a cloud-based server executing instructions out of a memory (e.g., 502, FIG. 5). The model ranks care providers (e.g., 506, FIG. 5).

In block B214, an API (e.g., the care pairing module 138) retrieves the rankings and pairs help seekers with care providers based on predictive positive conversation feedback. The machine learned model continues to be updated in block B216. The rankings are adjusted as more data is received and pairing may be modified based on the updated model.

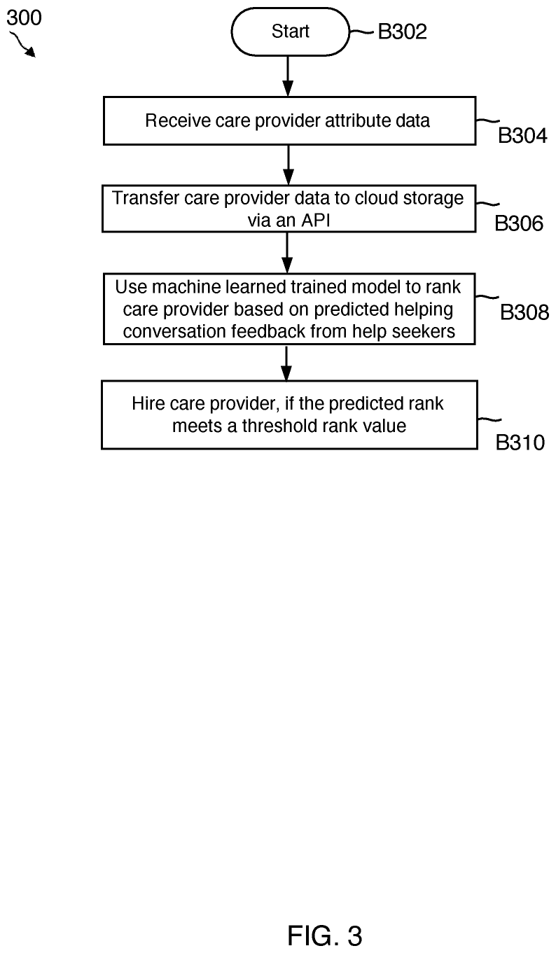

FIG. 3 shows a process 300 executed by module 136, according to one aspect of the present disclosure. The process begins in block B302 when the machine learned model has been developed. In block B304, care provider personality attributes (e.g., 510, FIG. 5) are received and then transferred to cloud based storage (e.g., to data structures 140) by an API in block B306. The attributes may be received by the host system 102 or directly by the micro-service 130.

In block B308, the machine learning regression model 504 is used to rank the care provider based on predicted helping conversation feedback from help seekers. If the rank meets a threshold value, then the care provider is hired in block B310.

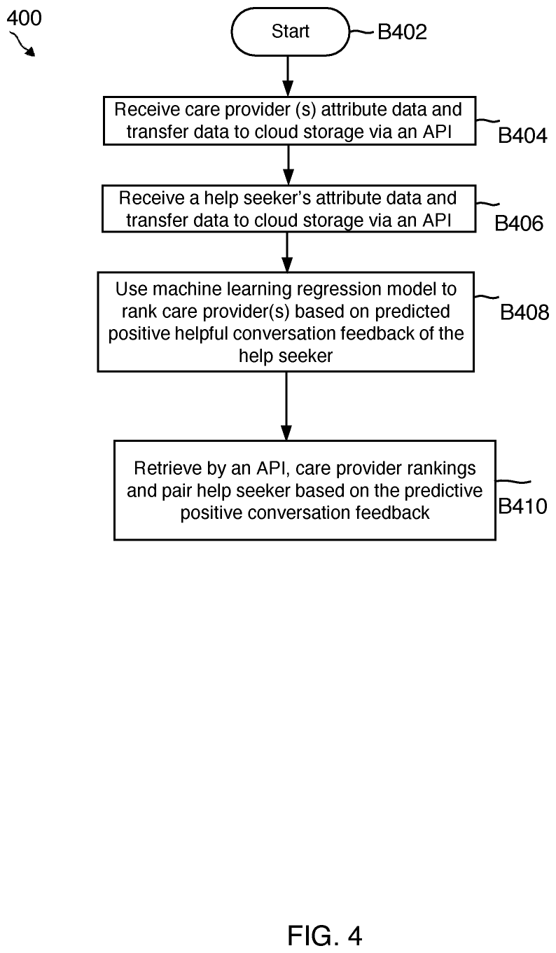

FIG. 4 shows a process 400 executed by module 138 for pairing a help seeker with care provider using a machine learned model (e.g., 504, FIG. 5), according to one aspect of the present disclosure. The process begins in block B402, when a computing device/cloud-based system has been initialized to execute the training module 134.

In block B404, care provider personality attributes (e.g., 510, FIG. 5) are received and then transferred to cloud-based storage (e.g., data structures 140) by an API. The attributes may be received by the host system 102 or directly by the micro-service 130.

In block B406, a help seeker's attributes (e.g., 508, FIG. 5) are received and transferred to cloud-based storage via an API. The attributes may be received by the host system 102 or directly by the micro-service 130.

In block B408, the machine learning regression model is used to rank care providers based on predicted positive helping conversation feedback of the help seeker.

In block B410, an API (e.g., module 138) retrieves the rankings and pairs the help seeker with a care provider based on predictive positive conversation feedback. The machine learned model continues to be updated. The rankings are adjusted as more data is received and the pairing may be modified based on the updated model.

Cloud Environment: FIG. 5 shows a cloud environment 500 to implement the various aspects of the present disclosure. The cloud environment 500 includes a cloud-based server 502 that has access to help seeker input 508 and care provider input 510. As described above with respect to FIGS. 2-4, the cloud-based server 502 executes the machine learned model 504 to generate care provider rankings 506 to pair help seekers with care providers based on the rankings.

As an example, inputs 508 and 510 may be collected through a Vue.js front end and a Node.js back end with data encoded and stored as JSON (JavaScript Object Notation) files. Amazon DynamoDB may be used as the database with ML model 504 written in Python and running through Amazon Sagemaker (without derogation of any Amazon trademark rights).

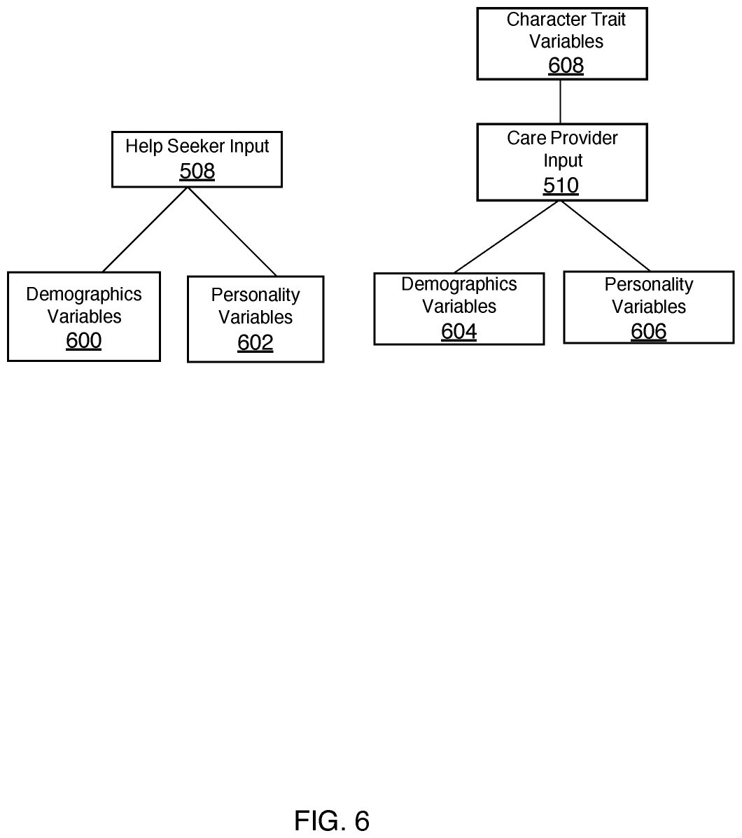

FIG. 6 shows an example of help seeker input 508 and care provider input 510.The help seeker input includes demographics data 600 and personality variables 602, while the care provider input includes demographics 604, personality variables 606 and character trait variables 608. These inputs can be stored in data structures 140 and used by the process blocks of FIGS. 2-4.

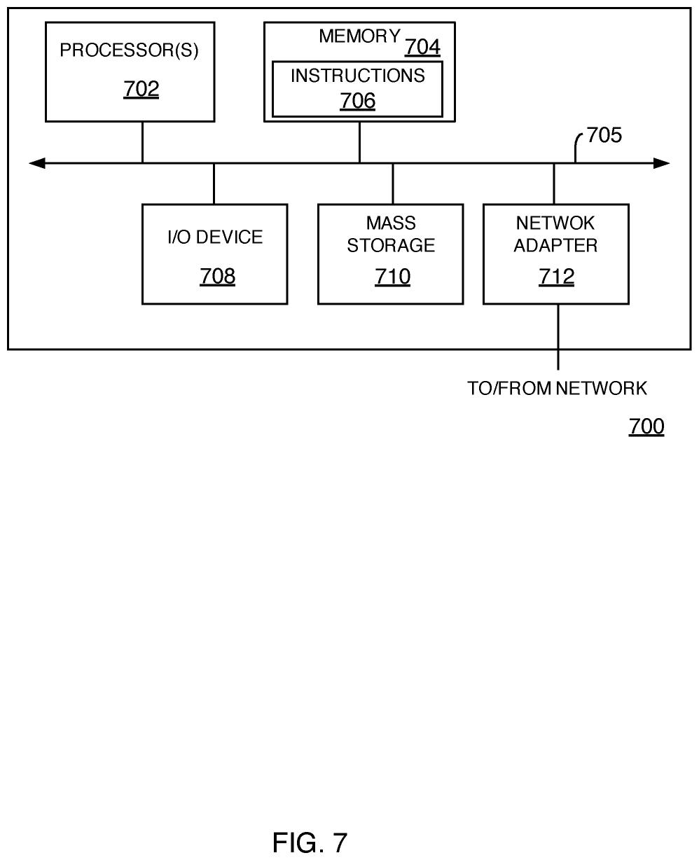

Processing System: FIG. 7 is a high-level block diagram showing an example of the architecture of a processing system 700 that may be used according to one aspect. The processing system 700 can represent host system 102, management system 118, user systems 116, cloud provider 122, storage system 108, and a cloud-based device (e.g., 502, FIG. 5) hosting the micro-services 130. Note that certain standard and well-known components which are not germane to the present aspects are not shown in FIG. 7.

The processing system 700 includes one or more processor(s) 702 and memory 704, coupled to a bus system 705. The bus system 705 shown in FIG. 7 is an abstraction that represents any one or more separate physical buses and/or point-to-point connections, connected by appropriate bridges, adapters and/or controllers. The bus system 705, therefore, may include, for example, a system bus, a Peripheral Component Interconnect (PCI) bus, a HyperTransport or industry standard architecture (ISA) bus, a small computer system interface (SCSI) bus, a universal serial bus (USB), or an Institute of Electrical and Electronics Engineers (IEEE) standard 1394 bus (sometimes referred to as “Firewire”).

The processor(s) 702 are the central processing units (CPUs) of the processing system 700 and, thus, control its overall operation. In certain aspects, the processors 702 accomplish this by executing software stored in memory 704. A processor 702 may be, or may include, one or more programmable general-purpose or special-purpose microprocessors, digital signal processors (DSPs), programmable controllers, application specific integrated circuits (ASICs), programmable logic devices (PLDs), or the like, or a combination of such devices.

Memory 704 represents any form of random access memory (RAM), read-only memory (ROM), flash memory, or the like, or a combination of such devices. Memory 704 includes the main memory of the processing system 700. Instructions 706 implement the process steps of FIGS. 2-4 and data structures 140 described above may reside in and execute (by processors 702) from memory 704.

Also connected to the processors 702 through the bus system 705 are one or more internal mass storage devices 710, and a network adapter 712. Internal mass storage devices 710 may be or may include any conventional medium for storing data in a non-volatile manner. The network adapter 712 provides the processing system 700 with the ability to communicate with remote devices (e.g., storage servers) over a network and may be, for example, an Ethernet adapter, a Fibre Channel adapter, or the like.

The processing system 700 also includes one or more input/output (I/O) devices 708 coupled to the bus system 705. The I/O devices 708 may include, for example, a display device, a keyboard, a mouse, etc.

Thus, innovative care paring technology in a cloud-based system has been described. Note that references throughout this specification to “one aspect” (or “embodiment”) or “an aspect” mean that a particular feature, structure or characteristic described in connection with the aspect is included in at least one aspect of the present disclosure. Therefore, it is emphasized and should be appreciated that two or more references to “an aspect” or “one aspect” or “an alternative aspect” in various portions of this specification are not necessarily all referring to the same aspect. Furthermore, the particular features, structures or characteristics being referred to may be combined as suitable in one or more aspects of the disclosure, as will be recognized by those of ordinary skill in the art.

While the present disclosure is described above with respect to what is currently considered its preferred aspects, it is to be understood that the disclosure is not limited to that described above. To the contrary, the disclosure is intended to cover various modifications and equivalent arrangements within the spirit and scope of the appended claims.

What is claimed is:

1. Methods and systems described herein.
